(19) United States
(12) Patent Application Publication
(10) Pub. No.: US 2017/0269000 A1
(43) Pub. Date: Sep. 21, 2017

(54) OBSERVATION SYSTEM AND OBSERVATION METHOD

(71) Applicant: OLYMPUS CORPORATION, Tokyo (JP)

(72) Inventor: Yoshioki KANEKO, Tokyo (JP)

(73) Assignee: OLYMPUS CORPORATION, Tokyo (JP)

(21) Appl. No.: 15/616,995

(22) Filed: Jun. 8, 2017

Related U.S. Application Data
(63) Continuation of application No. PCT/JP2014/082875, filed on Dec. 11, 2014.

(51) Int. Cl. G01N 21/64; G02B 21/00; A61B 5/00

(52) U.S. Cl. CPC G01N 21/6458; A61B 5/0071; G02B 21/0076; G02B 21/0032; G02B 21/0044; G02B 21/0048; G01N 2021/6478; G01N 2201/0636

(57) ABSTRACT

Provided is a system for observing an object that emits fluorescence when irradiated with excitation light. The system includes: a hole unit having holes on a plane perpendicular to an optical axis of the objective lens to allow the excitation light to pass through the holes in a direction parallel to the optical axis; and an imaging unit including: an imaging lens configured to focus the fluorescence; a microlens array having microlenses arranged on a plane perpendicular to an optical axis of the imaging lens; and an image sensor having pixels configured to: receive the fluorescence via the objective lens, at least one of the holes, and the microlens array, the fluorescence being emitted when the object is irradiated with the excitation light having passed through at least one of the holes and the objective lens; and output an image signal in accordance with an intensity of the received fluorescence.

### CROSS REFERENCES TO RELATED APPLICATIONS

This application is a continuation of PCT international application Ser. No. PCT/JP2014/082875 filed on Dec. 11, 2014 which designates the United States, incorporated herein by reference.

### BACKGROUND

1. Technical Field

The disclosure relates to a system and method for observing an object that emits fluorescence when the object is irradiated with excitation light.

2. Related Art

In related art, a fluorescence observation technique of irradiating a specimen with excitation light and observing fluorescence resulting from the irradiation is known. With the fluorescence observation technique, a specimen such as a biological cell is stained with a fluorescent substance, and fluorescence emitted by the fluorescent substance is detected, which allows the specimen to be observed at the molecular level.

In addition, a confocal observation technique of arranging pinholes on a plane (confocal plane) conjugate with a focal plane of the objective lens on the object side, detecting only fluorescence from the object plane, that is, focused fluorescence, and generating an image is also known. JP 2006-350005 A, for example, discloses a confocal microscope system that shifts the focal position of an objective lens to a specified Z position in the focus direction during an idle period at every cycle time of three-dimensional measurement, and displays a slice image of a specimen at the Z position.

In recent years, an observation device of the following system is also known, in which a disk-shaped member called a Nipkow disk having an arrangement of a plurality of pinholes is inserted on an optical path of illumination light, and the Nipkow disk is rotated in a plane perpendicular to the optical path, so that a specimen is irradiated at a plurality of points at the same time with illumination light rays having passed through the pinholes. JP 2008-233543 A, for example, discloses a confocal optical scanning detection device including a Nipkow disk having two types of pinholes with different diameters so that both of high resolution and bright field will be achieved. In addition, JP 2011-85759 A discloses a confocal optical scanner that changes the diameters of pinholes by inserting and removing a plurality of hole units having pinholes to/from an optical path of illumination light, the hole units being different from one another in the diameter of the pinholes.

### SUMMARY

In some embodiments, provided is a system for observing an object that emits fluorescence when the object is irradiated with excitation light via an objective lens. The system includes: a hole unit having a plurality of holes arranged on a plane perpendicular to an optical axis of the objective lens to allow the excitation light to pass through the plurality of holes in a direction parallel to the optical axis; and an imaging unit including: an imaging lens configured to focus the fluorescence; a microlens array having a plurality of microlenses arranged on a plane perpendicular to an optical axis of the imaging lens; and an image sensor having a plurality of pixels configured to: receive the fluorescence via the objective lens, at least one of the plurality of holes, and the microlens array, the fluorescence being emitted by the object when the object is irradiated with the excitation light having passed through the objective lens and at least one of the plurality of holes; and output an image signal in accordance with an intensity of the received fluorescence. The plurality of holes includes a plurality of types of holes different in pinhole position, the pinhole position being a position where a beam diameter of the excitation light passing through each hole is smallest in a direction of the optical axis of the objective lens. Each of the plurality of microlenses is configured to output the fluorescence incident on the plurality of microlenses via the imaging lens, in a direction depending on an incident direction of the fluorescence. The imaging unit is configured to: divide the received fluorescence to obtain divided fluorescence emissions according to a position of the at least one of the plurality of holes through which the fluorescence has passed, on the plane perpendicular to the optical axis of the objective lens; and output the image signal for each of the divided fluorescence emissions.

In some embodiments, provided is an observation method executed by an observation system for observing an object that emits fluorescence when the object is irradiated with excitation light via an objective lens. The method includes: irradiating the object with the excitation light via the objective lens and at least one of a plurality of holes, the plurality of holes being arranged on a plane perpendicular to an optical axis of the objective lens to allow the excitation light to pass through the plurality of holes in a direction parallel to the optical axis; and receiving, via the objective lens and at least one of the plurality of holes, the fluorescence emitted by the object when the object is irradiated with the excitation light, to output an image signal. The plurality of holes includes a plurality of types of holes different in pinhole position, the pinhole position being a position where a beam diameter of the excitation light passing through each hole is smallest in a direction of the optical axis of the objective lens. The receiving of the fluorescence and outputting of the image signal includes: receiving, by an image sensor, the fluorescence output from a microlens array, the microlens array having a plurality of microlenses arranged on a plane perpendicular to an optical axis of an imaging lens, each of the plurality of microlenses being configured to output the fluorescence incident on the plurality of microlenses via the imaging lens, in a direction depending on an incident direction of the fluorescence, the image sensor having a plurality of pixels configured to output the image signal in accordance with an intensity of the received fluorescence; dividing the received fluorescence to obtain divided fluorescence emissions according to a position of the at least one of the plurality of holes through which the fluorescence has passed, on the plane perpendicular to the optical axis of the objective lens; and outputting the image signal for each of the divided fluorescence emissions.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating an exemplary configuration of an observation system according to a first embodiment of the present invention;

FIG. 2 is a perspective view with a partial cross section illustrating a structure of a pinhole array illustrated in FIG. 1;

FIG. 3 is a schematic diagram illustrating an exemplary configuration of an imaging unit illustrated in FIG. 1;

FIG. 4 is a flowchart illustrating operation of an image processing device illustrated in FIG. 1;

FIG. 5 is a schematic diagram illustrating an image region expressed by image data based on an image signal output from the imaging unit illustrated in FIG. 3;

FIG. 6 is a schematic diagram for explaining a subject distance stored in a distance map;

FIG. 7 is a schematic graph for explaining a method for generating an image on a refocus plane;

FIG. 8 is a schematic diagram illustrating an example of a screen for a user to select a refocus plane;

FIG. 9 is a schematic diagram illustrating a structure of a pinhole array according to a modification of the first embodiment of the present invention;

FIG. 10 is a schematic diagram illustrating a configuration of an observation system according to a second embodiment of the present invention;

FIG. 11 is a schematic diagram illustrating a structure of a Nipkow disk illustrated in FIG. 10;

FIG. 12 is a schematic diagram illustrating a configuration of an observation system according to a third embodiment of the present invention;

FIG. 13 is a schematic diagram illustrating a structure of a microlens array illustrated in FIG. 12;

FIG. 14 is a schematic diagram illustrating a structure of a Nipkow disk illustrated in FIG. 12;

FIG. 15 is a schematic diagram illustrating a configuration of an observation system according to a fourth embodiment of the present invention; and

FIG. 16 is a schematic diagram illustrating an exemplary configuration of an endoscope system according to a fifth embodiment of the present invention.

### DETAILED DESCRIPTION

Exemplary embodiments of an observation system, optical components, and an observation method will be described in detail with reference to the drawings. The same reference numerals are used to designate the same elements throughout the drawings.

### First Embodiment

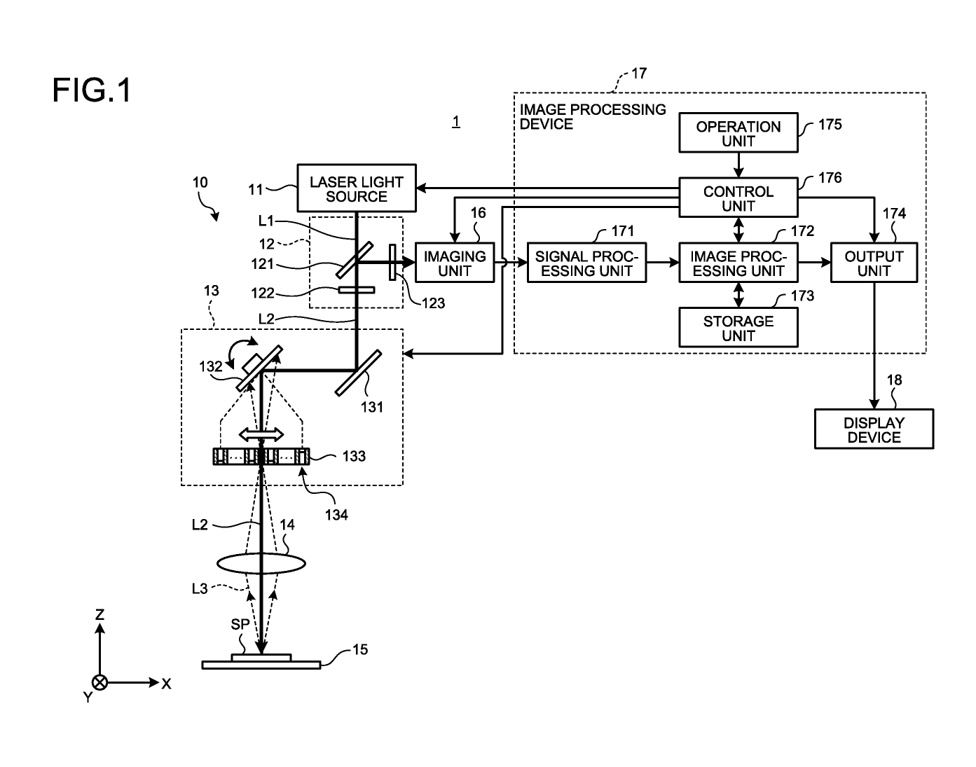

FIG. 1 is a schematic diagram illustrating an exemplary configuration of an observation system according to a first embodiment of the present invention. As illustrated in FIG. 1, an observation system 1 according to the first embodiment is a system for generating an image of a specimen SP that emits fluorescence when being irradiated with excitation light having a component within a specific wavelength band, and includes a microscope system 10 configured to generate and output an image signal relating to the specimen SP, an image processing device 17 configured to perform various processes on the image signal output from the microscope system 10, and a display device 18.

The microscope system 10 includes a laser light source 11 configured to emit laser light, a fluorescence unit 12 configured to extract the excitation light from laser light and extract fluorescence from light returning from the specimen SP, a hole unit 13 having a plurality of holes 134 through which the excitation light and the fluorescence passes, an objective lens 14 that collects the excitation light to irradiate the specimen SP with the excitation light and collects fluorescence emitted by the specimen SP, a stage 15 on which the specimen SP is placed, and an imaging unit 16 configured to capture an image of the fluorescence extracted by the fluorescence unit 12. In the following, the optical axis direction of the objective lens 14 will be referred to as a Z direction, and a plane perpendicular to the optical axis Z will be referred to as an XY plane.

The laser light source 11 emits laser light L1 having a component (excitation light) in a specific wavelength band capable of exciting the specimen SP. In the first embodiment, as will be described below, since the holes 134 are sequentially scanned by the excitation light extracted from the laser light L1, an ultrashort pulsed laser light source having a pulse period of one femtosecond or smaller is preferably used for the laser light source 11. The laser light source 11 emits laser light with a predetermined pulse period according to control performed a control unit 176 included in the image processing device 17, which will be described below.

The fluorescence unit 12 includes a dichroic mirror 121 that transmits a component containing the excitation light, of the laser light L1 incident from the direction of the laser light source 11, and reflects a component containing fluorescence, of light incident from the direction of the hole unit 13, toward the imaging unit 16, an excitation filter 122 that selectively transmits excitation light L2 from the component having passed through the dichroic mirror 121, and an absorption filter 123 that selectively transmits fluorescence from the component reflected by the dichroic mirror 121 and absorbs the other wavelength component.

The hole unit 13 includes a reflecting mirror 131, a galvanometer mirror 132, a pinhole array 133 in which a plurality of holes (through-holes) 134 are arranged. The reflecting mirror 131 reflects the excitation light having exited the fluorescence unit 12, so that the reflected excitation light is incident on the galvanometer mirror 132. The galvanometer mirror 132 is a mirror rotatable about an X axis and a Y axis, deflects the excitation light incident via the reflecting mirror 131 in a direction perpendicular to the XY plane, so that the excitation light sequentially passes through the holes 134. The pinhole array 133 is installed in a state in which a plane of arrangement of the holes 134 is parallel to the XY plane.

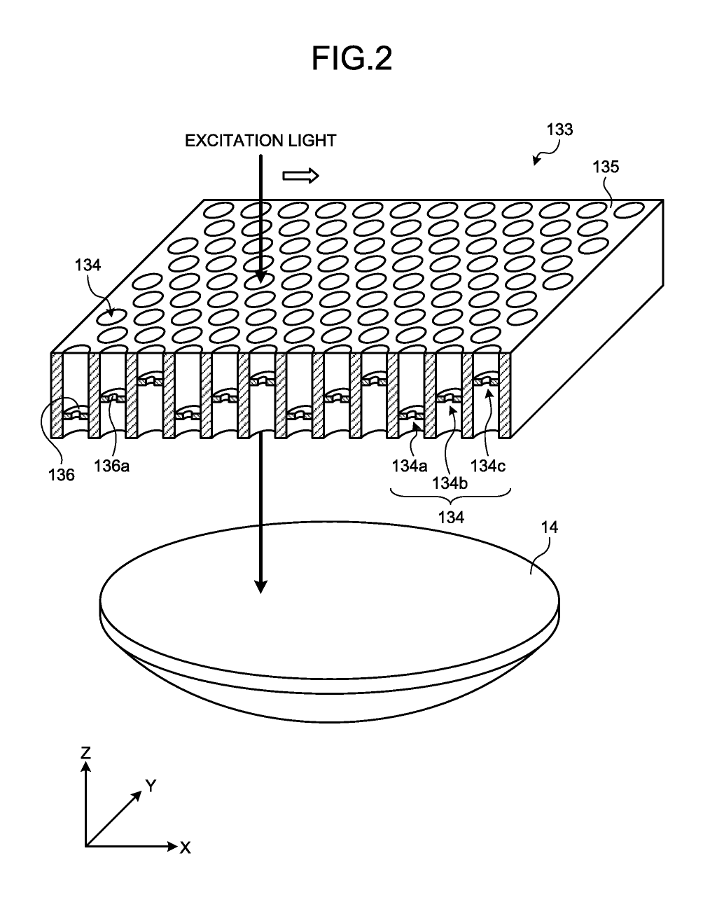

FIG. 2 is a perspective view with a partial cross section illustrating a structure of the pinhole array 133. The pinhole array 133 has a base material 135 in which the holes (through-holes) 134 are formed, and pinhole members 136 each disposed in a respective one of the holes 134. The base material 135 is formed of a light-blocking material such as metal or opaque synthetic resin. Each of the holes 134 has a columnar shape (a cylindrical shape, for example) and have a central axis perpendicular to a main surface of the base material 135.

The pinhole members 136 are disk-shaped (plate-shaped) members each having a through-hole (pinhole) 136a at the center, and made of a light-blocking material such as metal or opaque synthetic resin. The depth (the position in the thickness direction of the base material 135) at which each of the pinhole members 136 is fitted is set depending on the position of each hole 134 on the XY plane.

Note that light having entered each of the holes 134 passes through the pinhole 136a formed in the pinhole member 136 and exits the hole 134. Thus, the beam diameter of the light is smallest when the light passes through the pinhole 136a. Hereinafter, the position where the beam diameter of light (excitation light or fluorescence) having entered a hole 134 is smallest in the Z direction will be referred to as a pinhole position.

In FIG. 2, the pinhole members 136 are fitted at three pinhole positions. Hereinafter, the holes 134 may be classified into three types of holes 134a, 134b, and 134c depending on the pinhole positions. These holes 134a, 134b, and 134c are the same in the aperture diameter of the pinholes 136a but are different from one another in the distances between the pinhole positions and the objective lens 14. The distances between the pinhole positions of the holes 134a, 134b, and 134c and the objective lens 14 are not particularly limited, and can be adjusted by changing the position in the Z direction of the pinhole array 133 or the objective lens 14 as necessary. Note that the pinhole position of any of the holes 134a, 134b, and 134c may be adjusted to a focal plane of the objective lens 14.

In addition, the arrangement of the holes 134a, 134b, and 134c on the XY plane is not particularly limited, but it is preferable to arrange the respective types of holes 134a, 134b, and 134c as even as possible. In FIG. 2, the three types of holes 134a, 134b, and 134c are arranged in a successive order.

The hole unit 13 drives the galvanometer mirror 132 in synchronization with the pulse period of the laser light source 11 to scan the pinhole array 133 with the excitation light having exited the fluorescence unit 12, according to control performed by the control unit 176 included in the image processing device 17, which will be described below. In this manner, the excitation light L2 sequentially passes through any of the holes 134a, 134b, and 134c where the pinhole positions are different. In addition, the hole unit 13 deflects light L3 containing fluorescence, which have been emitted by the specimen SP, passed through the objective lens 14 and passed through any of the holes 134, by the galvanometer mirror 132 and the reflecting mirror 131, so that the deflected light L3 enters the fluorescence unit 12.

The description refers back to FIG. 1, in which the objective lens 14 focuses the excitation light L2 having exited the hole unit 13 onto the specimen SP, collects the light L3 containing fluorescence emitted by the specimen SP and makes the light L3 enter the hole unit 13.

The imaging unit 16 is a so-called light field camera (See Ren Ng et al., “Light Field Photography with a Hand-held Plenoptic Camera,” Stanford Tech Report CTSR, 2005-02), configured to separate images of fluorescence having entered the imaging unit 16 on the basis of the optical path of the fluorescence, that is, the position on the XY plane of one of the holes 134a, 134b, and 134c through which the fluorescence has passed, and records the images.

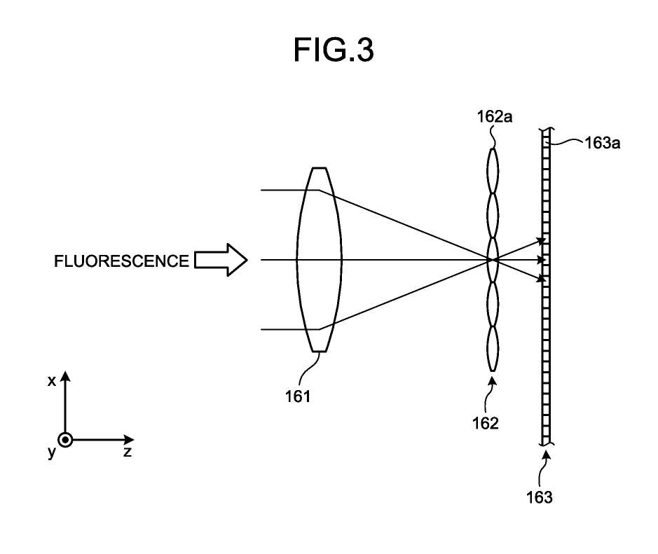

FIG. 3 is a schematic diagram illustrating an exemplary configuration of the imaging unit 16. The imaging unit 16 has an imaging lens 161 configured to focus the fluorescence incident on the imaging unit 16, a microlens array 162 disposed in parallel with the imaging lens 161, and an image sensor 163 disposed at the back of the microlens array 162 in parallel with the microlens array 162. In FIG. 3, the optical axis direction of the imaging lens 161 is referred to as a z direction, and a plane perpendicular to the z direction is referred to as an xy plane.

The imaging lens 161 is disposed so that the focal plane of the imaging lens 161 is conjugate with the focal plane of the objective lens 14. The microlens array 162 is disposed near the focal plane of the imaging lens 161.

The microlens array 162 has a plurality of microlenses 162a arranged two-dimensionally along the xy plane. The microlenses 162a outputs the fluorescence incident via the imaging lens 161 in a direction depending on the direction in which the fluorescence incident on the imaging lens 161 and the pupil region of the imaging lens 161 through which the fluorescence has passed. Thus, the imaging lens 161 and the microlens array 162 constitute a direction separating optical system that outputs fluorescence having entered the imaging unit 16 in a direction depending on the incident direction and the incidence position of the fluorescence, in other words, the position of the hole 134 through which the fluorescence has passed.

The image sensor 163 has a light receiving surface on which a plurality of pixels 163a are arranged two-dimensionally, and is constituted by a solid state image sensor such as a CCD or a CMOS. The image sensor 163 has an imaging function of forming a color image having a pixel level (pixel value) in each of R (red), G (green), and B (blue) bands, and operates at predetermined timing according to control performed by the control unit 176 of the image processing device 17, which will be described below.

The fluorescence having entered the imaging unit 16 is directed to a direction depending on the incident direction and the incidence position by the imaging lens 161 and the microlens array 162, and is incident on a pixel 163a at the position in this direction. The pixels 163a outputs electrical signals (image signals) on the basis of the intensity of the received light. Since the pixels 163a on which the fluorescence emissions having exited the respective microlenses 162a in the respective directions will be incident are preset, the optical path of fluorescence having entered the imaging unit 16 can be estimated from the image signals output from the pixels 163a of the image sensor 163.

The image processing device 17 includes a signal processing unit 171 configured to generate an image signal by processing an electrical signal output from the imaging unit 16, an image processing unit 172 configured to generate an image by performing predetermined image processing on the basis of the image signal generated by the signal processing unit 171, a storage unit 173 configured to store images generated by the image processing unit 172 and various other information data, an output unit 174, an operating unit 175 configured to receive input of instructions to the image processing device 17 and information, and the control unit 176 configured to generally control the respective units.

The signal processing unit 171 performs processing such as amplification and A/D conversion on electrical signals output from the imaging unit 16, and outputs digital image signals (hereinafter referred to as image data).

The image processing unit 172 performs processing such as white balance processing, demosaicing, color conversion, and gray level transformation (gamma conversion) on the image data output from the signal processing unit 171 to generate image data for display. The image processing unit 172 also generates images at a plane conjugate with pinhole positions of the respective holes 134a, 134b, and 134c provided in the pinhole array 133, that is, images of a plurality of different slices of the specimen SP on the basis of the image data, and performs compression processing of compressing the generated images, composition processing of generating a composite images of images of different slices, and the like. Furthermore, the image processing unit 172 may perform processing such as detection of an object region and association of coordinate information on the generated images or composite image.

The storage unit 173 is constituted by: a recording device or the like including a recording medium, such as a semiconductor memory such as an updatable flash memory, a RAM, or a ROM, a hard disk that is built in or connected via a data communication terminal, an MO, a CD-R, or a DVD-R; a recording device including a reading/writing device configured to writing/reading information into/from the recording medium; and the like. The storage unit 173 stores image data such as images at respective focal planes and composite images generated by the image processing unit 172, and other related information.

The output unit 174 is an external interface configured to output images of respective slices and composite images of these images generated by the image processing unit 172, user interface screens, and the like to external devices such as the display device 18 under the control of the control unit 176.

The operating unit 175 includes an input device such as a keyboard, various buttons, and various switches, a pointing device such as a mouse and a touch panel, and is configured to input a signal according to an operation externally performed by a user to the control unit 176.

The control unit 176 generally controls operation of the entire observation system 1 on the basis of various instructions and various information data input from the operating unit 175.

Note that the image processing unit 172 and the control unit 176 may be constituted by dedicated hardware or may be implemented by reading a predetermined program in hardware such as a CPU. In the latter case, the storage unit 173 further stores control programs for controlling the operation of the observation system 1, image processing programs to be executed by the image processing unit 172, various parameters and setting information used in execution of the programs, and the like.

The display device 18 is constituted by an LCD, an EL display, or a CRT display, for example, and is configured to display an image or the like output from the image processing device 17.

Next, the operation of the observation system 1 will be described. First, the observation system 1 is powered on, and a specimen SP is placed on the stage 15. Under the control of the control unit 176, the laser light source 11 is then caused to emit laser light L1 with a predetermined pulse period, and the galvanometer mirror 132 is driven in synchronization with the pulse period of the laser light L1. Thus, excitation light L2 extracted from the laser light via the fluorescence unit 12 sequentially passes through the holes 134 provided in the pinhole array 133. The excitation light L2 having passed through the holes 134 is collected by the objective lens 14 for irradiation of an object plane of the specimen SP to cause the specimen SP to emit fluorescence. The fluorescence (see light L3) is collected by the objective lens 14, passes through the holes 134 through which the excitation light L2 has previously passed, and enters the imaging unit 16 via the fluorescence unit 12. Thus, an image signal expressing an image of the fluorescence is output from the imaging unit 16 to the image processing device 17.

In the series of operation, control is performed so that the excitation light L2 passes through every one of the holes 134 once within one exposure period (within one frame period) of the imaging unit 16. This means that the fluorescence emissions emitted from regions of the specimen SP corresponding to the positions of the holes 134 arranged in the pinhole array 133 enter the imaging unit 16 within one exposure period. In other words, image information on the regions of the specimen SP corresponding to the entire plane of arrangement of the holes 134 can be obtained within one exposure period.

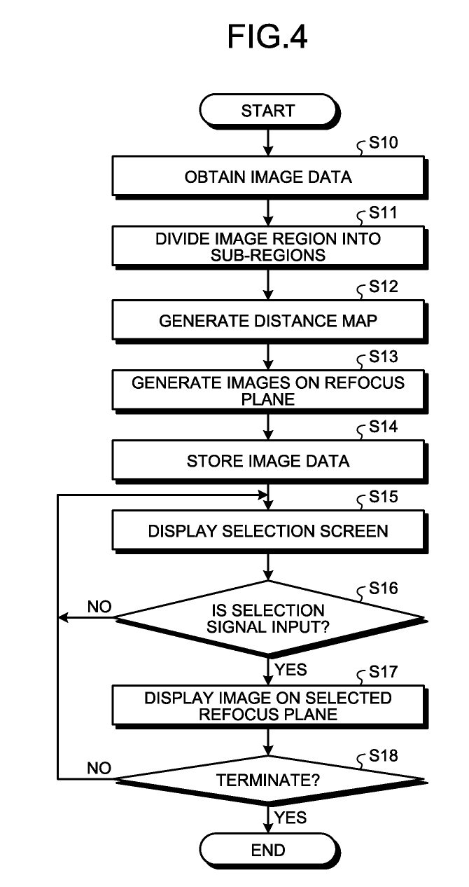

FIG. 4 is a flowchart illustrating the operation of the image processing device 17 after an image signal is received. First, in step S10, the image processing device 17 performs processing such as amplification and A/D conversion on the image signal output from the imaging unit 16 to generate image data, and further performs processing such as white balance processing, demosaicing, color conversion, and gray level transformation (gamma conversion) on the image data to obtain image data for display.

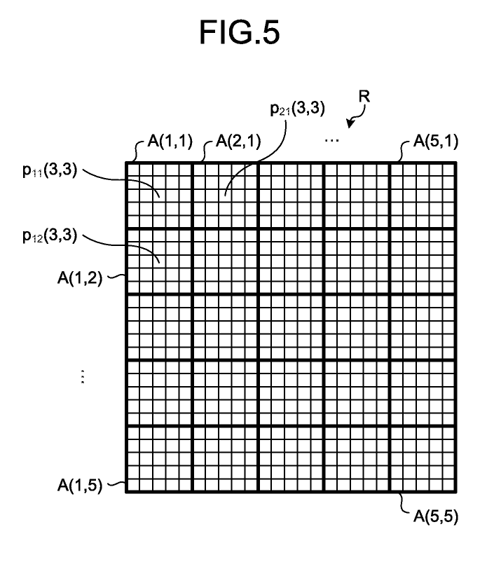

FIG. 5 is a schematic diagram illustrating an image region R expressed by image data based on an image signal output from the imaging unit 16. The position of the pixels constituting the image region R correspond to the positions of the pixels 163a arranged on the light receiving surface of the image sensor 163.

In step S11, the image processing unit 172 divides the image region R into a plurality of sub-regions according to the arrangement of the microlenses 162a in the microlens array 162 (see FIG. 3). A symbol A(m,n) in FIG. 5 represents the position of a sub-region in the image region R. For example, in a case where a total of 25 microlenses 162a, which are five in the x direction and five in the y direction, are arranged in the microlens array 162, the image region R is similarly divided into 5×5=25 sub-regions (m=1 to 5, n=1 to 5). Thus, information on fluorescence output from one microlens 162a is recorded in one sub-region A(m,n) in the image region R.

As described above, fluorescence having entered the imaging unit 16 is incident on a microlens 162a at a position in a direction depending on the direction in which the fluorescence is incident on the imaging lens 161 and the incidence position (pupil region), and is further incident on a pixel 163a at a position in a direction depending on the direction in which the fluorescence is incident on the microlens 162a. Thus, through extraction of pixels where information on one common pupil region is stored (pixels pₘₙ(3,3) at the centers of the respective sub-regions A(m,n), for example) from among the sub-regions A(m,n) in the image region R and computation using the pixel values of the extracted pixels, an image focused on a virtual plane (also called a refocus plane) different from the focal plane (the plane of arrangement of the microlens array 162) of the imaging lens 161 can be formed (See Ren Ng et al, “Light Field Photography with a Hand-held Plenoptic Camera,” Stanford Tech Report CTSR, 2005-02, for the principle of a light field camera and an image configuration on a refocus plane).

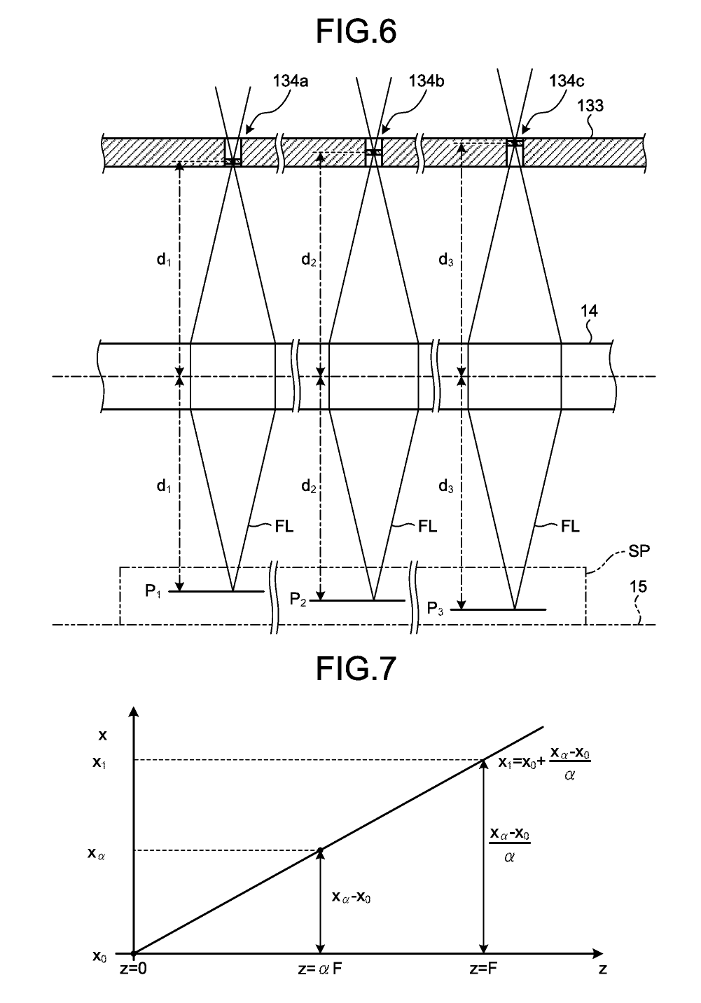

In subsequent step S12, the image processing unit 172 generates a distance map in which each of the pixels in the image region R and a subject distance (a distance between the objective lens 14 and an object plane) on an optical path through which the fluorescence incident on the pixel has passed are associated with each other. FIG. 6 is a schematic diagram for explaining a subject distance stored in the distance map. In FIG. 6, for the purpose of illustration, the ratios of the respective elements are different from those in FIG. 1.

As illustrated in FIG. 6, any one of the plurality of types of holes 134a, 134b, and 134c, which are different in pinhole position, is located on each optical path of fluorescence FL emitted by the specimen SP. Thus, planes conjugate with the pinhole positions in the holes 134a, 134b, and 134c through which fluorescence has passed are object planes P₁, P₂, and P₃. Thus, the distances between the pinhole positions in the holes 134a, 134b, and 134c through which fluorescence has passed and the objective lens 14 are given as subject distances d₁, d₂, and d₃.

In addition, since the positions of the pixels in the image region R are associated with the positions of the pixels 163a of the image sensor 163 and since an optical path of fluorescence incident on each of the pixels 163a (the position of a hole 134 through which fluorescence has passed) is determined from the positional relation of the imaging lens 161 and each of the microlenses 162a, the pixels in the image region R and the subject distances d₁, d₂, and d₃ can be associated on the basis of the positional relation.

In subsequent step S13, the image processing unit 172 generates images of fluorescence generated at the object planes P₁, P₂, and P₃ on the basis of the distance map generated in step S12.

FIG. 7 is a schematic graph for explaining a method for generating an image on a refocus plane. As illustrated in FIG. 3, fluorescence emitted by the specimen SP is incident on the imaging lens 161 and focused onto the microlens array 162. In FIG. 7, a coordinate in the optical axis (z axis) direction of the imaging lens 161 is z=0, a coordinate of the focal plane of the imaging lens 161 is z=F, and a coordinate of an image plane (refocus plane) conjugate with an object plane (any one of the object planes P₁, P₂, and P₃) is z=αF (0<α<1). Note that the focal plane of the imaging lens 161 corresponds to the plane of arrangement of the microlens array 162, and to the plane conjugate with the focal plane of the objective lens 14. A coefficient α is a coefficient for determining the coordinate of the refocus plane, and is given as a ratio of a subject distance d₁, d₂, or d₃ of each object plane P₁, P₂, or P₃ to the focal distance of the objective lens 14.

Although a method of calculating a pixel value in the x direction of an image at the refocus plane will be described below for better understanding, a pixel value can be similarly calculated in the y direction.

In the following, coordinates (x,z) of a pupil region of the imaging lens 161 are denoted by (x₀,0), and fluorescence having passed through the pupil region has passed through a point (x_{α},αF) on the refocus plane and reached a point (x₁,F) on the focal plane. The x coordinate x₁ of the focal plane at this point is given by an expression (1) below.

x₁=x₀+(x_{α}−x₀)/α (1)

When an output value (intensity of fluorescence) of the pixel 163a on which the fluorescence having passed through the pupil region (x=x₀) and the point (x=x₁) on the focal plane is incident is represented by I(x₀,x₁), an output value I_{α}(x_{α}) at a point x_{α} on the refocus plane is obtained by integration of I(x₀,x₁) with the pupil region of the imaging lens 161, and given by an expression (2) below.

$${I}_{α} ( {x}_{α} ) = \frac{1}{{( α F )}^{2}} \int I ( {x}_{0} , {x}_{0} + \frac{{x}_{α} - {x}_{0}}{α} ) {dx}_{0} \qquad ( 2 )$$

Since the focal distance F and the coefficient α in the expression (2) are given, the microlens 162a (coordinate x=x₁) on which the fluorescence is incident is determined by the expression (1) if the pupil region x=x₀ through which the fluorescence has passed and the desired point x_{α} on the refocus plane are given. Then, the pixel 163a on which the fluorescence having passed through the pupil region x=x₀ is incident is determined from the arrangement of the pixel 163a on which the fluorescence having passed through the determined microlens 162a is incident. The output value of the pixel 163a is equal to the aforementioned output value (x₀,x₁). Thus, the pixel values of the pixels constituting an image on a refocus plane I_{α}(x) can be calculated by computation of integrating an output value of a pixel 163a with a pupil region for all of the pupil regions of the imaging lens 161. When the pupil region x=x₀ is a representative coordinate of the pupil regions of the imaging lens 161, the expression (2) can be rewritten as an expression of simple addition.

In this manner, an image on a refocus plane can be obtained by calculation of pixel values of the pixels constituting the image on the refocus plane. The image processing unit 172 generates images on refocus planes corresponding to the object planes P₁, P₂, and P₃. In this process, the image processing unit 172 may further combine the images on the refocus planes corresponding to the object planes P₁, P₂, and P₃ to generate a 3D image or an all-in-focus image.

In subsequent step S14, the image processing unit 172 stores image data of the images generated in step S13 in the storage unit 173.

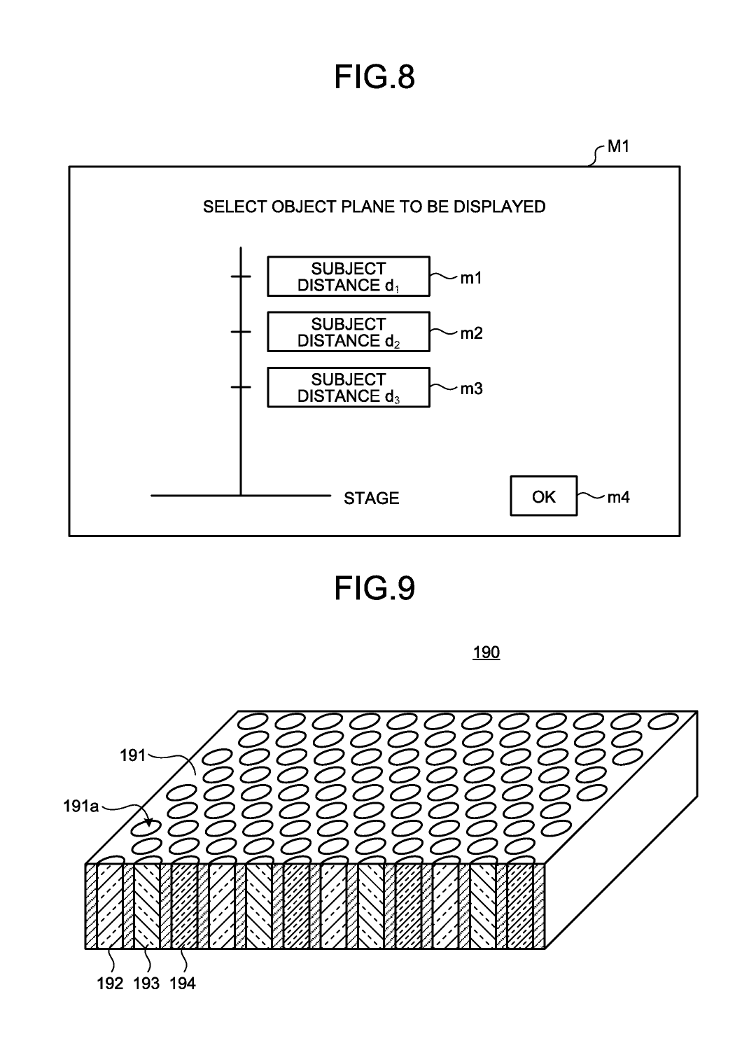

In subsequent step S15, the control unit 176 displays, on the display device 18, a screen (selection screen) for the user to select a refocus plane to be displayed on the display device 18. FIG. 8 is a schematic diagram illustrating an example of a screen for the user to select a refocus plane. A screen M1 illustrated in FIG. 8 includes icons m1 to m3 respectively showing the subject distances d₁, d₂, and d₃ of the object planes P₁, P₂, and P₃ corresponding to the refocus planes on which images are generated, and an OK button m4.

In subsequent step S16, the control unit 176 determines whether or not a selection signal for selecting one of the refocus planes has been input from the operating unit 175. When one of the icons m1 to m3 is selected by a pointing operation on the screen M1 with an input device such as a mouse and an operation on the OK button m4 is made, for example, a selection signal for selecting the refocus plane corresponding to the subject distance of the selected icon is input. Note that, when a 3D image or an all-in-focus image is generated in step S13, display of the 3D image or all-in-focus image may also be selectable options in addition to the refocus planes.

If the selection signal for selecting one of the refocus planes has been input (step S16: Yes), the control unit 176 outputs the input selection signal to the image processing unit 172, and causes the display device 18 to output the image data of the selected refocus plane from the image processing unit 172 via the output unit 174 to display the image on the display device 18 (step S17). If a 3D image or an all-in-focus image is generated in step S13 and a selection signal for selecting display of one of these images is input in step S16, the control unit 176 causes the display device 18 to display the selected image.

If no selection signal has been input (step S16: No), the control unit 176 continues display of the selection screen (step S15) and waits until any selection signal is input. Alternatively, while waiting for the input, the control unit 176 may display the image on a predetermined specific refocus plane on the display device 18. Specifically, examples of the image include an image on a refocus plane at a subject distance closest to the focal distance of the objective lens 14, an image on a refocus plane at a middle subject distance (the subject distance d₂ in the case of FIG. 6, for example), an image on a refocus plate at the shortest subject distance (the subject distances d₁ in the case of FIG. 6, for example), and an image on a refocus plane at the longest subject distance (the subject distances d₃ in the case of FIG. 6, for example).

In step S18, the control unit 176 determines whether or not a signal indicating termination of the observation system is input from the operating unit 175. If no signal indicating the termination is input (step S18: No), the operation of the control unit 176 returns to step S15. If a signal indicating the termination is input (step S18: Yes), the control unit 176 terminates the operation of the observation system 1.

As described above, in the first embodiment of the present invention, fluorescence having passed through the plurality of types of holes 134a, 134b, and 134c which are different in pinhole position, and having been incident on the imaging unit 16 within one imaging period is divided in directions depending on the incident directions and the incidence positions of the fluorescence, and recorded in the pixels 163a located in the directions in which the fluorescence is divided. Thus computation using output values of the pixels 163a allows slice images of the specimen SP on the planes conjugate with the pinhole positions to be generated by one imaging operation. Thus, even in observation of a biological specimen, a plurality of images focused on respective slices with high accuracy are obtained with no positional displacement caused in the XY plane. In addition, a 3D image or an all-in-focus image can also be formed by combining these images.

In the related art, for generation of images of a plurality of slices, since imaging is repeated while the focal plane is gradually shifted with respect to a specimen, the specimen is repeatedly exposed to excitation light, which causes a problem that the fluorescence stain applied to the specimen is likely to fade. In contrast, in the first embodiment, since images of a plurality of slices are generated on the basis of image signals obtained by one imaging operation, the time during which a specimen SP is exposed to excitation light is shortened, and it is also possible to suppress fading of fluorescence stain applied to the specimen SP.

In addition, according to the first embodiment of the present invention, since an ultrashort pulsed laser light source 11 of a femtosecond or shorter is used, a deep portion (on the order of several hundred μm) of a biological specimen can also be observed.

Furthermore, according to the first embodiment of the present invention, since the base material 135 having a plurality of holes 134 formed therein is produced and the pinhole members 136 are fitted at different depths in the holes 134 so that the pinhole positions are varied, the pinhole aperture diameters and the pinhole positions can be readily controlled with high accuracy.

While three pinhole positions are used in the pinhole array 133 in the first embodiment, the number of pinhole positions is not limited thereto. Specifically, two or four or more pinhole positions may be used. The coefficient α in formation of images on refocus planes by the image processing unit 172 may be set according to the pinhole positions.

Modification

Next, a modification of the first embodiment of the present invention will be described.

While the pinhole array 133 having the pinhole members 136 fitted at different depths in the holes 134 formed in the base material 135 is used in the first embodiment, the configuration of a pinhole array that can be used in the hole unit 13 is not limited thereto. FIG. 9 is a schematic diagram illustrating a structure of a pinhole array according to the modification.

A pinhole array 190 illustrated in FIG. 9 includes a base material 191 in which a plurality of holes 191a are formed, and optical members 192, 193, and 194 with which the insides of the holes 191a are filled. The optical members 192, 193, and 194 are transparent members capable of transmitting excitation light and fluorescence and have different refractive indices from one another. Excitation light having been made to enter any of the holes 191a by the galvanometer mirror 132 and fluorescence collected by the objective lens 14 are converged onto a position in the Z direction depending on the refractive index of the optical member with which the hole 191a entered by the excitation light is filled. Thus, the optical members 192, 193, and 194 with which the holes 191a are filled functions similarly to the pinholes. In the present application, in a case where light is converged by an optical member in this manner, the position on the optical path where the beam diameter of the light is smallest is referred to as a pinhole position.

While the holes 191a are filled with any of three types of optical members 192, 193, and 194 in a successive order so that three pinhole positions are set in the modification, the number of pinhole positions is not limited to three but may be two or four or larger. The refractive indices of the optical members may be appropriately selected on the basis of the pinhole positions to be set.

According to the modification, the pinhole array 190 having different types of holes with different pinhole positions is easily produced with high accuracy.

### Second Embodiment

Next, a second embodiment of the present invention will be described.

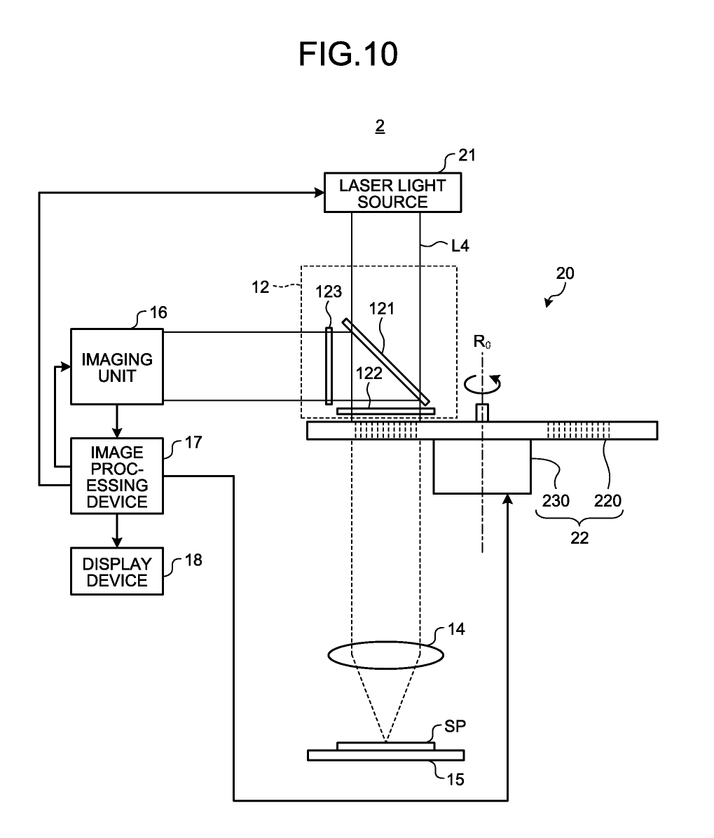

FIG. 10 is a schematic diagram illustrating a configuration of an observation system according to the second embodiment of the present invention. As illustrated in FIG. 10, an observation system 2 according to the second embodiment includes a microscope system 20, an image processing device 17 configured to perform various processes on an image signal output from the microscope system 20, and a display device 18. Among these elements, the configurations and the operations of the image processing device 17 and the display device 18 are similar to those in first embodiment.

The microscope system 20 includes a laser light source 21 instead of the laser light source 11 illustrated in FIG. 1, and includes a hole unit 22 instead of the hole unit 13 illustrated in FIG. 1. The configurations of the elements of the microscope system 20 other than the laser light source 21 and the hole unit 22 are similar to those of the microscope system 10 illustrated in FIG. 1.

The laser light source 21 is a pulsed laser light source having a component (excitation light) in a wavelength band capable of exciting a specimen SP similarly to the laser light source 11, but emits laser light L4 having a beam diameter larger than that of the laser light source 11.

The hole unit 22 includes a Nipkow disk 220 having a plurality of type of holes with different pinhole positions, and a motor 230 configured to rotate the Nipkow disk 220 about a rotational axis R₀.

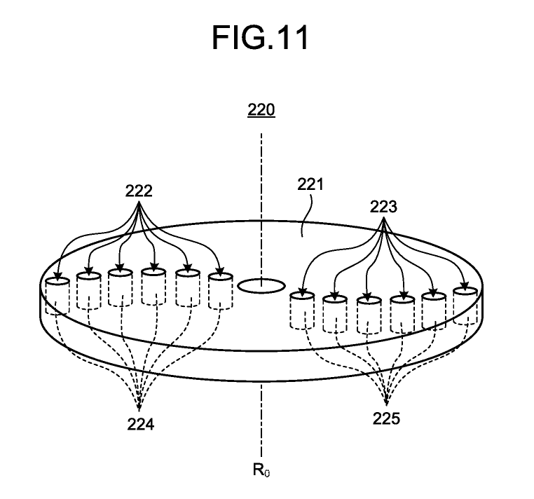

FIG. 11 is a schematic diagram illustrating a structure of the Nipkow disk 220. The Nipkow disk 220 includes a disk-shaped base material 221 in which a plurality of holes 222 and 223 are formed, optical members 224 with which the holes 222 are filled, and optical members 225 with which the holes 223 are filled. The holes 222 and 223 are arranged spirally on a main surface of the base material 221. While one spiral line of the holes 222 and one spiral line of the holes 223 are formed in FIG. 11, a plurality of spiral lines of the respective holes may be formed.

The optical members 224 and 225 are transparent members capable of transmitting excitation light and fluorescence and have different refractive indices from each other. The excitation light and the fluorescence having entered either of the holes 222 and 223 are converged onto a position (pinhole position) depending on the refractive index of the optical member with which the hole is filled.

For imaging of a specimen SP, laser light L4 is emitted from the laser light source 21 in a pulsed manner, and the Nipkow disk 220 is rotated at a predetermined speed by the motor 230 in synchronization with the pulse period. As a result, excitation light having exited the fluorescence unit 12 enters a plurality of holes (the holes 222 or 223 or the both) at the same time, passes through the optical members (the optical members 224 or 225) with which the holes entered by the excitation light are filled, and is converged once. Thereafter, the excitation light expands again and is collected by the objective lens 14, so that the specimen SP is irradiated at a plurality of points at the same time. In addition, fluorescence emitted at the plurality of points of the specimen SP passes through the objective lens 14, enters a plurality of holes (the holes 222 or 223 or the both) of the Nipkow disk 220, is once converged by the optical members (the optical members 224 or 225) with which the holes entered by the fluorescence are filled, then expands again, and enters the imaging unit 16 via the fluorescence unit 12.

In the series of operation, control is performed so that the holes 222 and 223 formed in the Nipkow disk 220 cover over the entire cross-sectional region of the laser light L4 emitted by the laser light source 21 within one exposure period of the imaging unit 16. Thus, image information on the regions of the specimen SP corresponding to the entire cross-sectional region of the laser light L4 can be obtained within one exposure period.

According to the configuration in the second embodiment of the present invention as described above, since a specimen SP can be irradiated with excitation light at a plurality of points (multibeam irradiation), a specimen SP can be imaged in a shorter time than in the first embodiment. Thus, positional displacement of a specimen SP in the XY plane can further be reduced in three-dimensional information on the specimen SP.

Furthermore, according to the second embodiment of the present invention, the holes 222 and 223 formed in the base material 221 are filled with optical members 224 and 225, respectively, having different refractive indices, which allows the Nipkow disk 220 having a plurality of pinhole positions to be easily produced with high accuracy.

### Third Embodiment

Next, a third embodiment of the present invention will be described.

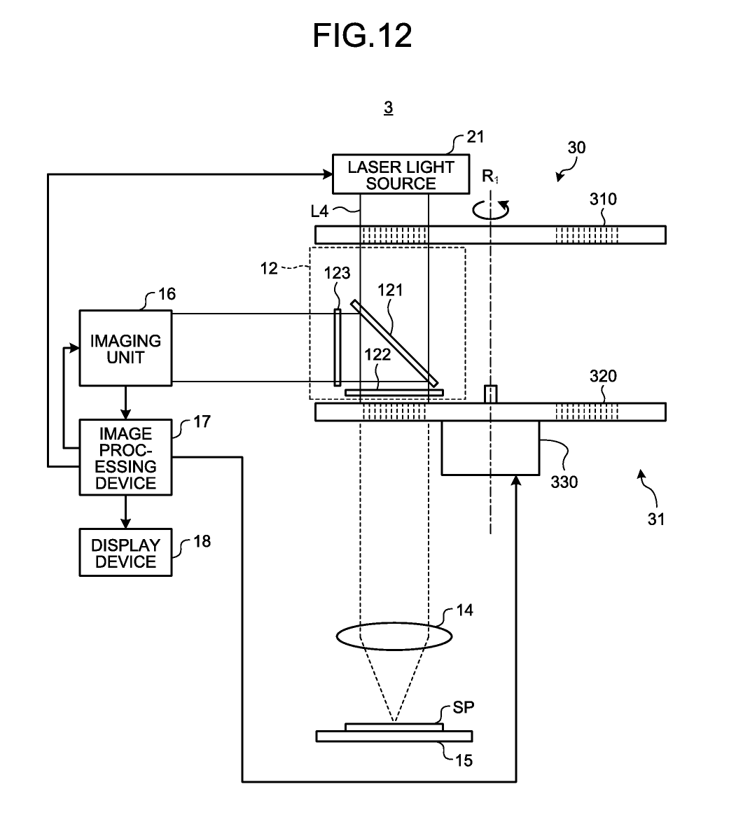

FIG. 12 is a schematic diagram illustrating a configuration of an observation system according to the third embodiment of the present invention. As illustrated in FIG. 12, an observation system 3 according to the third embodiment includes a microscope system 30, an image processing device 17 configured to perform various processes on an image signal output from the microscope system 30, and a display device 18. Among these elements, the configurations and the operations of the image processing device 17 and the display device 18 are similar to those in first embodiment.

The microscope system 30 includes a hole unit 31 instead of the hole unit 22 illustrated in FIG. 10. The configurations of the elements of the microscope system 30 other than the hole unit 31 are similar to those of the microscope system 20 illustrated in FIG. 10.

The hole unit 31 includes a microlens array 310 and a Nipkow disk 320 arranged in parallel to each other, and a motor 330 configured to rotate the microlens array 310 and the Nipkow disk 320 about a rotational axis R₁.

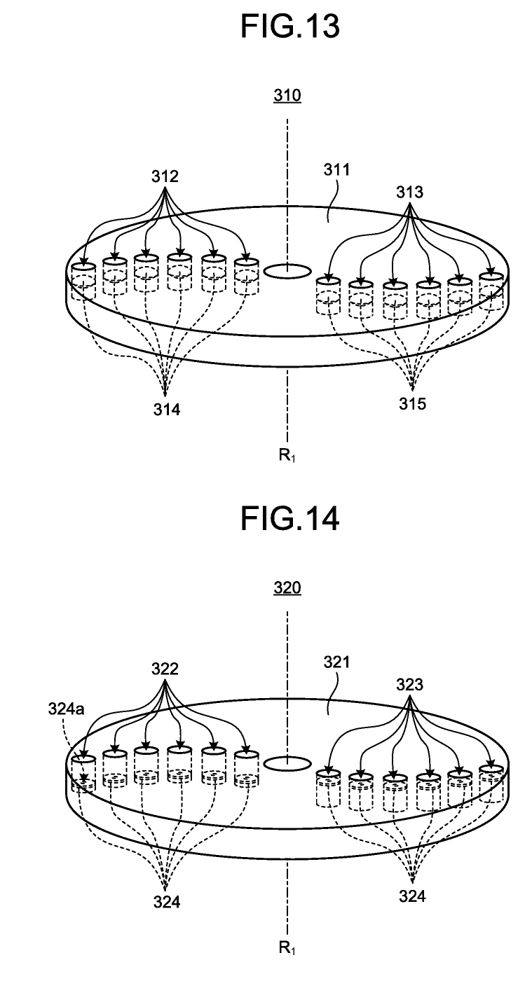

FIG. 13 is a schematic diagram illustrating a structure of the microlens array 310. The microlens array 310 includes a disk-shaped base material 311 in which a plurality of holes 312 and 313 are formed, microlenses 314 fitted into the holes 312, and microlenses 315 fitted into the holes 313. The holes 312 and 313 are arranged spirally on a main surface of the base material 311. While one spiral line of the holes 312 and one spiral line of the holes 313 are formed in FIG. 13, a plurality of spiral lines of the respective holes may be formed.

The microlenses 314 and 315 are made of optical members having different refractive indices from each other. The excitation light and the fluorescence having entered either of the holes 312 and 313 are focused onto a focal plane depending on the refractive index of the microlens 314 or 315 fitted in the hole.

FIG. 14 is a schematic diagram illustrating a structure of the Nipkow disk 320. Nipkow disk 320 includes a base material 321 in which a plurality of holes 322 and 323 are formed, and pinhole members 324 fitted into the holes 322 and 323. The pinhole members 324 are disk-shaped members each having a through-hole (pinhole) 324a at the center, and made of a light-blocking material such as metal or opaque synthetic resin. The pinhole members 324 are fitted into the holes 322 and 323, where the depths at which the pinhole members 324 are fitted are different between the holes 322 and the holes 323. While one spiral line of the holes 322 and one spiral line of the holes 323 are formed in FIG. 14, a plurality of spiral lines of the respective holes may be formed so that the numbers of spiral lines correspond to those of the holes 312 and 313 of the microlens array 310.

The microlens array 310 and the Nipkow disk 320 are arranged in parallel to each other with the fluorescence unit 12 therebetween, in such a manner that the holes 312 are opposed to the holes 322 and that the holes 313 are opposed to holes 323. In addition, the distance between the microlens array 310 and the Nipkow disk 320 is set so that the focal points of the microlenses 314 are coincident with the pinhole positions of the opposed holes 322 and that the focal points of the microlenses 315 are coincident with the pinhole positions of the holes 323. As a result, excitation light extracted from laser light collected by the microlenses 314 and 315 is converged onto the pinhole positions of the holes 322 and 323 and passes through the holes 322 and 323.

For imaging of a specimen SP, laser light L4 is emitted from the laser light source 21 in a pulsed manner, and the microlens array 310 and the Nipkow disk 320 are rotated together by the motor 330 in synchronization with the pulse period. As a result, laser light is collected by the microlenses formed in the microlens array 310, and excitation light enters the holes of the Nipkow disk 320 at the same time via the fluorescence unit 12. The excitation light is once converged onto the pinhole positions of the holes which the excitation light has entered, then expands again, and is collected by the objective lens 14, so that the specimen SP is irradiated with the excitation light at a plurality of positions at the same time. In addition, fluorescence emitted at the plurality of points of the specimen SP passes through the objective lens 14, enters a plurality of holes of the Nipkow disk 320, is once converged onto the pinhole positions of the holes which the fluorescence has entered, then expands again, and enters the imaging unit 16 via the fluorescence unit 12.

In the series of operation, control is performed so that the holes 312 and 313 formed in the microlens array 310 and the holes 322 and 323 formed in the Nipkow disk 320 cover over the entire cross-sectional region of the laser light L4 emitted by the laser light source 21 within one exposure period of the imaging unit 16. Thus, image information on the regions of the specimen SP corresponding to the entire cross-sectional region of the laser light L4 can be obtained within one exposure period.

According to the configuration in the third embodiment as described above as well, since a specimen SP can be irradiated with excitation light at a plurality of points (multibeam irradiation), a specimen SP can be imaged in a shorter time than in the first embodiment. Thus, positional displacement of a specimen SP in the XY plane can further be reduced in three-dimensional information on the specimen SP. In addition, according to the third embodiment, since a specimen SP can be irradiated with more intense excitation light as a result of using the microlenses 314 and 315, a clearer image of fluorescence can be obtained.

Furthermore, in the third embodiment, since the microlenses 314 and 315 are formed with use of optical materials having different refractive indices, a microlens array disk on which different types of microlenses with different focal distances are arranged can be easily produced with high accuracy.

### Fourth Embodiment

Next, a fourth embodiment of the present invention is described.

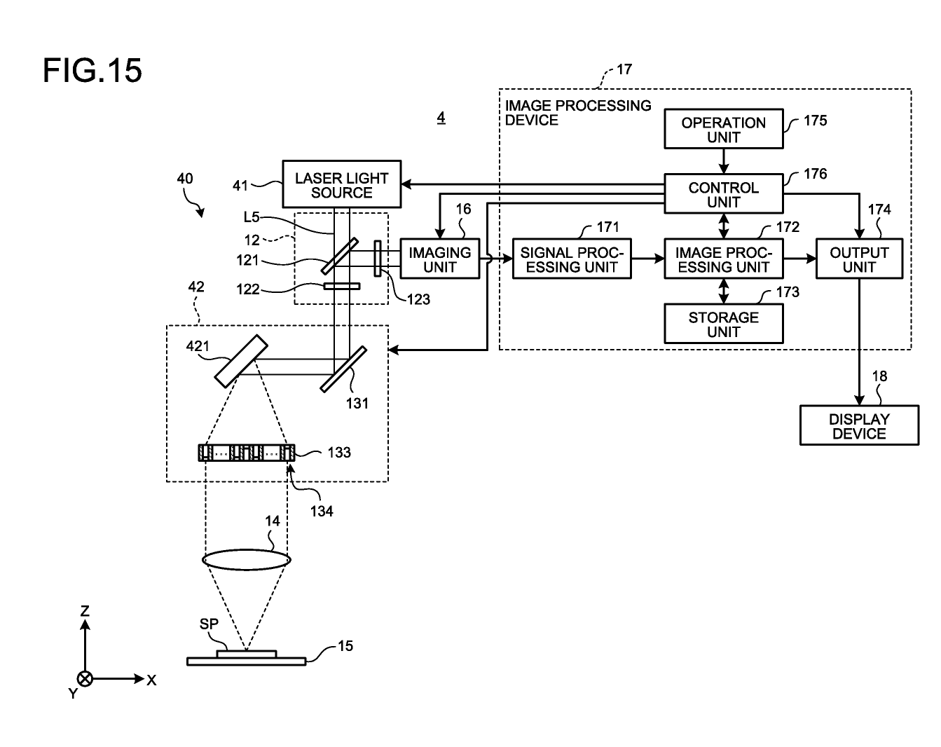

FIG. 15 is a schematic diagram illustrating a configuration of an observation system according to the fourth embodiment of the present invention. As illustrated in FIG. 15, an observation system 4 according to the fourth embodiment includes a microscope system 40, an image processing device 17 configured to perform various processes on an image signal output from the microscope system 40, and a display device 18. Among these elements, the configurations and the operations of the image processing device 17 and the display device 18 are similar to those in first embodiment.

The microscope system 40 includes a laser light source 41 instead of the laser light source 11 illustrated in FIG. 1, and includes a hole unit 42 instead of the hole unit 13 illustrated in FIG. 1. The configurations of the elements of the microscope system 40 other than the laser light source 41 and the hole unit 42 are similar to those of the microscope system 10 illustrated in FIG. 1.

The laser light source 41 is a pulsed laser light source having a component (excitation light) in a wavelength band capable of exciting a specimen SP similarly to the laser light source 11, but emits laser light L5 having a beam diameter larger than that of the laser light source 11.

The hole unit 42 includes a reflecting mirror 131, a digital mirror device (DMD) 421, and a pinhole array 133. Among these elements, the reflecting mirror 131 and the pinhole array 133 have the same configurations as those in the first embodiment.

The digital mirror device 421 is an MEMS device provided with a plurality of micromirrors and capable of on-off control of reflecting function. The micromirrors are arranged in directions in which the micromirrors can reflect excitation light incident via the reflecting mirror 131 toward the respective holes 134 formed in the pinhole array 133. The pinholes are grouped into groups of several successive pinholes, and controlled so that the reflecting function is turned on and off in units of groups.

For imaging of a specimen SP, laser light L5 is emitted from the laser light source 41 in a pulsed manner, and the micromirrors provided on the digital mirror device 421 are sequentially turned on in units of groups in synchronization with the pulse period. As a result, the excitation light reflected by the micromirrors that have been turned on passes through corresponding holes 134, and is collected by the objective lens 14, so that a specimen SP is irradiated with the excitation light at a plurality of points at the same time. In addition, fluorescence emitted at the plurality of points of the specimen SP passes through the holes 134 at the same time via the objective lens 14, and enters the imaging unit 16 via the micromirrors having been turned on and the fluorescence unit 12.

In the series of operation, the grouping and the on-off control of the micromirrors are performed so that the excitation light having exited the fluorescence unit 12 passes through every one of the holes 134 once within one exposure period of the imaging unit 16. In other words, image information on the regions of the specimen SP corresponding to the entire plane of arrangement of the holes 134 can be obtained within one exposure period.

According to the configuration in the fourth embodiment described above, since the holes 134 into which the excitation light is to enter can be switched according to electronic control, a specimen SP can be imaged in a further shorter time than in the first to third embodiments. Thus, positional displacement of a specimen SP in the XY plane can further be reduced in three-dimensional information on the specimen SP.

### Fifth Embodiment

Next, a fifth embodiment of the present invention is described.

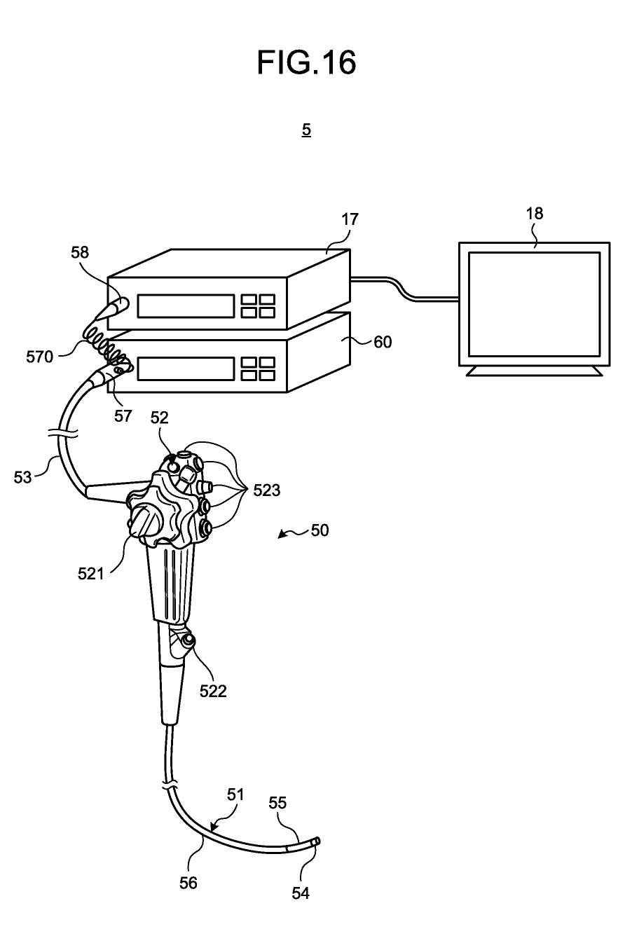

FIG. 16 is a schematic diagram illustrating an exemplary configuration of an endoscope system according to the fifth embodiment of the present invention. An endoscope system 5 illustrated in FIG. 16 is an embodiment of the observation system illustrated in FIG. 1, and includes an endoscope 50 to be inserted in a body of a subject and being configured to perform imaging to generate an image signal, a light source unit 60 configured to emit illumination light from a distal end of the endoscope 50, an image processing device 17 configured to generate an image on the basis of the image signal generated by the endoscope 50, and a display device 18 configured to display the image generated by the image processing device 17. Among these elements, the configurations and the operations of the image processing device 17 and the display device 18 are similar to those in first embodiment. In addition, the light source unit 60 is a pulsed light source containing excitation light, and emits laser light having a beam diameter larger than that of the laser light source 11.

The endoscope 50 includes a flexible insertion part 51 having an elongated shape, an operating unit 52 connected to a proximal end side of the insertion part 51 and configured to receive input of various operation signals, and universal cord 53 extending from the operating unit 52 in a direction opposite to the direction in which the insertion part 51 extends and including various cables connected to the image processing device 17 and the light source unit 60.

The insertion part 51 includes a distal end portion 54, a bendable bending portion 55 constituted by a plurality of bending pieces, and an elongated, flexible needle tube 56 connected to the proximal end side of the bending portion 55. The distal end portion 54 of the insertion part 51 is provided with the fluorescence unit 12, the hole unit 42, the objective lens 14, and the imaging unit 16 (see FIG. 15). Note that, as long as the objective lens 14 is provided in the distal end portion 54, the fluorescence unit 12, the hole unit 42, and the imaging unit 16 may be provided on either of the distal end portion 54 side and the operating unit 52 side. For example, among these elements, the objective lens 14 may be provided in the distal end portion 54, and the fluorescence unit 12, the hole unit 42, and the imaging unit 16 may be provided on the operating unit 52 side.

A cable assembly of a plurality of signal lines through which electrical signals are transmitted to and received from the image processing device 17 and a light guide for transmission of light are connected between the operating unit 52 and the distal end portion 54. The signal lines include a signal line for transmission of image signals output from the image sensor 163 (see FIG. 3) to the image processing device 17, a signal line for transmission of control signals output from the image processing device 17 to the image sensor 163, and the like.

The operating unit 52 includes a bending nob 521 for bending the bending portion 55 upward, downward, leftward, and rightward, a treatment tool insertion part 522 through which a treatment tool such as a biopsy needle, biopsy forceps, a laser knife, or an inspection probe is configured to be inserted, and a plurality of switches 523 which constitute an operation input unit configured to input operation instruction signals for peripheral devices such as an air conveyance unit, a water conveyance unit, and a gas conveyance unit in addition to the image processing device 17 and the light source unit 60.

The universal cord 53 includes at least the light guide and the cable assembly. In addition, a connector unit 57 attachable to and detachable from the light source unit 60, and an electric connector unit 58 being electrically connected to the connector unit 57 via a coil-shaped coil cable 570 and being attached to and detached from the image processing device 17 are provided at an end of the universal cord 53 opposite to the side connected to the operating unit 52. An image signal output from the image sensor 163 is input to the image processing device 17 via the coil cable 570 and the electric connector unit 58.

While an example in which the observation system 4 illustrated in FIG. 15 is applied to an endoscope system for a living body has been presented in the fifth embodiment described above, the observation systems 1, 2, and 3 illustrated in FIGS. 1, 10, and 12 may also be applied to an endoscope system. Furthermore, these observation systems 1 to 4 may also be applied to an industrial endoscope system.

According to some embodiments, fluorescence is emitted by an object when the object is irradiated with excitation light. The fluorescence passes through at least one of different types of holes, which are different in pinhole position, and is incident on the imaging unit. The fluorescence is divided to obtain divided fluorescence emissions according to the position of a hole through which the fluorescence has passed, and an image signal is output for each of the divided fluorescence emissions. This makes it possible to acquire three-dimensional image information at a desired part of a specimen with high accuracy.

The present invention is not limited to the first to fifth embodiments and the modification as described above, but the elements disclosed in the first to fifth embodiments and the modification can be appropriately combined to achieve various inventions. For example, some of the elements presented in the first to fifth embodiments and the modification may be excluded. Alternatively, elements presented in different embodiments may be appropriately combined.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A system for observing an object that emits fluorescence when the object is irradiated with excitation light via an objective lens, the system comprising:
a hole unit having a plurality of holes arranged on a plane perpendicular to an optical axis of the objective lens to allow the excitation light to pass through the plurality of holes in a direction parallel to the optical axis; and
an imaging unit including:
an imaging lens configured to focus the fluorescence;
a microlens array having a plurality of microlenses arranged on a plane perpendicular to an optical axis of the imaging lens; and
an image sensor having a plurality of pixels configured to: receive the fluorescence via the objective lens, at least one of the plurality of holes, and the microlens array, the fluorescence being emitted by the object when the object is irradiated with the excitation light having passed through the objective lens and at least one of the plurality of holes; and output an image signal in accordance with an intensity of the received fluorescence, wherein
the plurality of holes includes a plurality of types of holes different in pinhole position, the pinhole position being a position where a beam diameter of the excitation light passing through each hole is smallest in a direction of the optical axis of the objective lens,
each of the plurality of microlenses is configured to output the fluorescence incident on the plurality of microlenses via the imaging lens, in a direction depending on an incident direction of the fluorescence, and
the imaging unit is configured to:
divide the received fluorescence to obtain divided fluorescence emissions according to a position of the at least one of the plurality of holes through which the fluorescence has passed, on the plane perpendicular to the optical axis of the objective lens; and
output the image signal for each of the divided fluorescence emissions.

2. The system according to claim 1, wherein
the hole unit comprises:
a pinhole array having the plurality of holes arranged on the plane perpendicular to the optical axis of the objective lens; and
a galvanometer mirror configured to cause the excitation light to sequentially enter each of the plurality of holes, wherein
each of the plurality of holes includes a pinhole member at a depth corresponding to the pinhole position, the pinhole member being a plate-shaped light-blocking member having a through-hole.

3. The system according to claim 1, wherein
the hole unit comprises:
a pinhole array having the plurality of holes arranged on the plane perpendicular to the optical axis of the objective lens; and
a galvanometer mirror configured to cause the excitation light to sequentially enter each of the plurality of holes, wherein
each of the plurality of holes is filled with an optical member having a refractive index depending on the pinhole position.

4. The system according to claim 1, wherein
the hole unit comprises:
a Nipkow disk having a disk shape and having a main surface on which the plurality of holes is arranged; and
a drive unit configured to rotate the Nipkow disk about an axis parallel to the optical axis of the objective lens, wherein
each of the plurality of holes is filled with an optical member having a refractive index depending on the pinhole position.

5. The system according to claim 1, wherein
the hole unit comprises:
a Nipkow disk having a disk shape and having a main surface on which the plurality of holes is provided;
a lens array disk having a disk shape and having a lens arrangement surface parallel to the main surface of the Nipkow disk, the lens array disk having, on the lens arrangement surface, a plurality of lenses configured to collect the excitation light onto the plurality of holes; and
a drive unit configured to rotate the Nipkow disk and the lens array disk synchronously with each other about an axis parallel to the optical axis of the objective lens, wherein
each of the plurality of holes includes a pinhole member at a depth corresponding to the pinhole position, the pinhole member being a plate-shaped light-blocking member having a through-hole.

6. The system according to claim 5, wherein
each of the plurality of lenses is formed of an optical member having a refractive index depending on the pinhole position in each of the plurality of holes onto which the excitation light is collected.

7. The system according to claim 1, wherein
the hole unit comprises:
a pinhole array having the plurality of holes arranged on the plane perpendicular to the optical axis of the objective lens; and
a digital mirror device configured to cause the excitation light to enter some of the plurality of holes, wherein
each of the plurality of holes includes a pinhole member at a depth corresponding to the pinhole position, the pinhole member being a plate-shaped light-blocking member having a through-hole, and
the digital mirror device is configured to sequentially switch between the plurality of holes so as to cause the excitation light to enter some of the plurality of holes.

8. The system according to claim 1, wherein
the hole unit comprises:
a pinhole array having the plurality of holes arranged on the plane perpendicular to the optical axis of the objective lens; and
a digital mirror device configured to cause the excitation light to enter some of the plurality of holes, wherein
each of the plurality of holes is filled with an optical member having a refractive index depending on the pinhole position, and
the digital mirror device is configured to sequentially switch between the plurality of holes so as to cause the excitation light to enter some of the plurality of holes.

9. The system according to claim 1, further comprising:
a laser light source configured to emit ultrashort pulsed laser light having a pulse period of a femtosecond or smaller; and
a fluorescence unit configured to extract the excitation light from the ultrashort pulsed laser light emitted by the laser light source, and extract the fluorescence from light incident on the fluorescence unit via the objective lens and the at least one of the plurality of holes from the object.

10. An observation method executed by an observation system for observing an object that emits fluorescence when the object is irradiated with excitation light via an objective lens, the method comprising:
irradiating the object with the excitation light via the objective lens and at least one of a plurality of holes, the plurality of holes being arranged on a plane perpendicular to an optical axis of the objective lens to allow the excitation light to pass through the plurality of holes in a direction parallel to the optical axis; and
receiving, via the objective lens and at least one of the plurality of holes, the fluorescence emitted by the object when the object is irradiated with the excitation light, to output an image signal, wherein
the plurality of holes includes a plurality of types of holes different in pinhole position, the pinhole position being a position where a beam diameter of the excitation light passing through each hole is smallest in a direction of the optical axis of the objective lens, wherein
the receiving of the fluorescence and outputting of the image signal includes:
receiving, by an image sensor, the fluorescence output from a microlens array, the microlens array having a plurality of microlenses arranged on a plane perpendicular to an optical axis of an imaging lens, each of the plurality of microlenses being configured to output the fluorescence incident on the plurality of microlenses via the imaging lens, in a direction depending on an incident direction of the fluorescence, the image sensor having a plurality of pixels configured to output the image signal in accordance with an intensity of the received fluorescence;
dividing the received fluorescence to obtain divided fluorescence emissions according to a position of the at least one of the plurality of holes through which the fluorescence has passed, on the plane perpendicular to the optical axis of the objective lens; and
outputting the image signal for each of the divided fluorescence emissions.

11. The observation method according to claim 10, further comprising generating a plurality of images respectively corresponding to different pinhole positions of the plurality of holes, based on the image signal.
